(19) United States
(12) Patent Application Publication
(10) Pub. No.: US 2024/0416153 A1
(43) Pub. Date: Dec. 19, 2024

(54) BEAUTY MEDICAL DEVICE

(71) Applicant: HIRONIC CO., LTD., Yongin-si (KR)

(72) Inventors: Ae Rie JI, Yongin-si (KR); Jin Woo LEE, Yongin-si (KR); Kyong Su JUNG, Gunpo-si (KR); Jin Woo YU, Anyang-si (KR)

(73) Assignee: HIRONIC CO., LTD., Yongin-si (KR)

(21) Appl. No.: 18/817,209

(22) Filed: Aug. 27, 2024

Related U.S. Application Data
(63) Continuation of application No. 17/285,670, filed on Apr. 15, 2021.

(30) Foreign Application Priority Data
Oct. 15, 2018 (KR) 10-2018-0122700

(51) Int. Cl. A61N 7/02; A61B 18/00; A61B 18/14; A61N 1/08; A61N 1/40; A61N 7/00

(52) U.S. Cl. CPC A61N 7/02; A61B 18/14; A61N 1/08; A61N 1/403; A61B 2018/0047; A61B 2018/00589; A61B 2018/00994; A61N 2001/083; A61N 2007/0008; A61N 2007/0034

(57) ABSTRACT

According to one aspect of the present invention, provided is a beauty medical device including: a handpiece body having an ultrasonic emission surface prepared at one end; an ultrasonic generation unit which is provided inside the handpiece body and which generates an ultrasonic wave so as to emit same at the skin through the ultrasonic emission surface; and a high frequency generation unit disposed on the circumference of the ultrasonic emission surface at one end of the handpiece body so as to generate a high frequency.

### CROSS REFERENCE TO PRIOR APPLICATION

This application is a continuation application of U.S. patent application Ser. No. 17/285,670 filed on Apr. 15, 2021, which is a National Stage Application of PCT International Patent Application No. PCT/KR2019/013239 filed on Oct. 8, 2019, under 35 U.S.C. § 371, which claims priority to Korean Patent Application No. 10-2018-0122700 filed on Oct. 15, 2018, which are all hereby incorporated by reference in their entirety.

### BACKGROUND OF THE INVENTION

The present invention relates to a beauty medical device configured to perform both ultrasonic and radio frequency treatments using one device so as to reduce a treatment time and to improve a skin-lift effect or an adipolysis effect.

It is a recent trend that an interest in skin care and obesity care has increased day by day, and a variety of beauty medical devices for skin care and obesity care have been developed. For example, a variety of beauty medical devices for face lifting, skin tightening treatment, or adipolysis treatment have been developed.

In noninvasive treatment methods for skin care or obesity, there are a method using a radio frequency (RF), a method of using a vibratory message function, a method of using cryolipolysis, a method of focused ultrasonic waves, and the like.

As described above, a variety of noninvasive treatment methods may be selectively performed according to a state and condition of a person to be treated, and beauty medical devices for perform respective treatment methods have been studied and developed.

### RELATED ART DOCUMENT

### Patent Document

Korean Patent Registration No. 10-1574951 (Dec. 7, 2015)

### SUMMARY OF THE INVENTION

The present invention is directed to providing a beauty medical device configured to reduce a treatment time and improve a skin lifting effect or an adipolysis effect using a handpiece capable of performing both ultrasonic treatment and radio frequency treatment.

One aspect of the present invention provides a beauty medical device including a handpiece body including an ultrasonic emission surface at one end, an ultrasonic generation unit installed in the handpiece body and configured to generate an ultrasonic wave and emit the ultrasonic wave toward skin through the ultrasonic emission surface, and a radio frequency generation unit disposed at the one end of the handpiece body on a periphery of the ultrasonic emission surface and configured to generate a radio frequency.

The radio frequency generation unit may include a plurality of radio frequency electrodes arranged on the periphery of the ultrasonic emission surface.

The ultrasonic emission surface may have a circular shape.

The radio frequency generation unit may include an annular radio frequency electrode surrounding the ultrasonic emission surface.

The radio frequency generation unit may include a plurality of radio frequency electrodes arranged to be radially symmetrical on the basis of the ultrasonic emission surface.

The ultrasonic emission surface may include a linear portion having a quadrangular shape and a pair of semicircular portions having a semicircular shape and disposed on both ends of the linear portion to be symmetrical to each other.

The radio frequency generation unit may include a plurality of radio frequency electrodes arranged to be symmetrical on the basis of a virtual central line longitudinally crossing the ultrasonic emission surface.

The beauty medical device may further include a contact sensor configured to sense whether the one end of the handpiece body comes into contact with the skin and a control unit configured to operate the ultrasonic generation unit or the radio frequency generation unit when it is sensed by the contact sensor that the one end of the handpiece body comes into contact with the skin.

The beauty medical device may further include a printed circuit board coupled to the one end of the handpiece body. Here, the contact sensor may include a contact sensor electrode formed on one surface of the printed circuit board, and the radio frequency generation unit may include a radio frequency electrode formed on the other surface of the printed circuit board.

The radio frequency electrode may be disposed at a position corresponding to the contact sensor electrode.

The ultrasonic generation unit and the radio frequency generation unit may alternately operate.

The beauty medical device may further include a cooling unit configured to cool heat generated by at least one of the ultrasonic generation unit and the radio frequency generation unit.

The beauty medical device may further include a control unit configured to operate the cooling unit when a temperature inside the handpiece body exceeds a preset temperature.

A beauty medical device according to the present invention may reduce a treatment time and improve a skin lifting effect and an adipolysis effect using a handpiece capable of performing both ultrasonic treatment and radio frequency treatment.

### BRIEF DESCRIPTION OF THE DRAWINGS

FIGS. 1A and 1B are perspective views illustrating a beauty medical device according to the present invention.

FIG. 2 is a cross-sectional view illustrating the beauty medical device according to the present invention.

FIGS. 3A, 3B, and 3C are views illustrating an ultrasonic emission surface and a radio frequency electrode according to one embodiment of the present invention.

FIGS. 4 to 6 are views illustrating a contact sensor electrode and a radio frequency electrode according to one embodiment of the present invention.

FIGS. 7A, 7B, and 7C are views illustrating an ultrasonic emission surface and a radio frequency electrode according to another embodiment of the present invention.

FIGS. 8 to 9 are views illustrating a contact sensor electrode and a radio frequency electrode according to another embodiment of the present invention.

FIG. 10 is a graph illustrating ultrasonic wave and radio frequency generation periods according to one embodiment of the present invention.

### DETAILED DESCRIPTION OF THE INVENTION

Since the present invention may be variously modified and have a variety of embodiments, particular embodiments will be illustrated in the drawings and described in detail hereinafter. However, these are not intended to limit the present invention to a particularly disclosed form and it should be understood that the present invention includes all changes, equivalents, and substitutes included within the concept and technical scope of the present invention. In a description of the embodiments of the present invention, a detailed description of well-known components or functions of the related art will be omitted when it is deemed to obscure understanding of the embodiments of the present invention.

The terms such as first, second, and the like may be used to describe a variety of components, and the components are not limited to the terms. The terms are used only for distinguishing one component from another.

The terms used herein are used merely for describing particular embodiments and are not intended to limit the present invention. Singular expressions, unless clearly defined otherwise in context, include plural expressions. Throughout the application, the term “comprise,” “have,” or the like is used herein to specify the presence of stated features, numbers, stages, operations, elements, components or combinations thereof but do not preclude the presence or addition of one or more other features, numbers, stages, operations, elements, components, or combinations thereof.

Hereinafter, an embodiment of a beauty medical device 100 according to the present invention will be described in detail with reference to the attached drawings. In a description with reference to the attached drawings, like or corresponding elements will be referred to as like reference numerals and a repetitive description thereof will be omitted.

According to one embodiment of the present invention, there is provided the beauty medical device 100 including a handpiece body 110 including an ultrasonic emission surface 122 at one end, an ultrasonic generation unit 120 installed in the handpiece body 110 and configured to generate an ultrasonic wave and emit the ultrasonic wave toward skin through the ultrasonic emission surface 122, and a radio frequency generation unit 130 disposed at the one end of the handpiece body 110 on a periphery of the ultrasonic emission surface 122 and configured to generate a high frequency, that is, a radio frequency (RF).

According to the present invention, the beauty medical device 100 includes both the ultrasonic generation unit 120 and the radio frequency generation unit 130 so as to simultaneously or alternately perform ultrasonic treatment and radio frequency treatment.

Accordingly, when the ultrasonic treatment and the radio frequency treatment are performed using the beauty medical device 100 according to the present invention, an overall treatment time may be reduced and a skin lifting effect or an adipolysis effect may be improved. Also, since both the ultrasonic treatment and the radio frequency treatment may be performed using one handpiece, a practitioner may conveniently perform treatment.

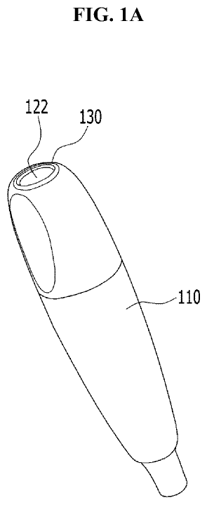

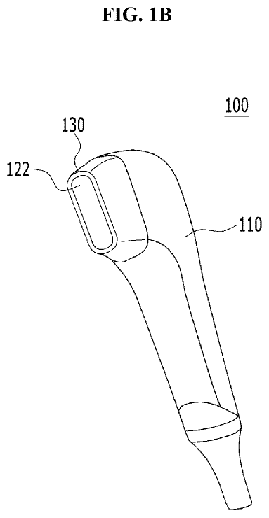

Hereinafter, respective components of the beauty medical device 100 according to the embodiment will be described with reference to FIGS. 1 to 10.

As shown in FIG. 1, the ultrasonic emission surface 122 may be provided at the one end of the handpiece body 110. In detail, the handpiece body 110 may include a cartridge portion configured to generate an ultrasonic wave and a radio frequency and a body portion in which components necessary for operating the cartridge portion are arranged and which has a structure grippable by the practitioner. The ultrasonic emission surface 122 may be formed on the cartridge portion at the one end of the handpiece body 110.

Also, since it is necessary to replace the cartridge portion when a certain number of ultrasonic waves are generated, the cartridge portion may be detachably coupled to the body portion.

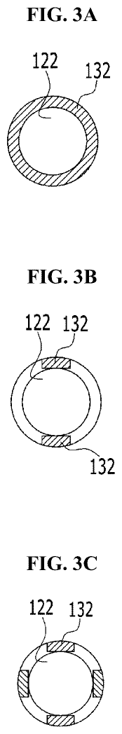

As shown in FIGS. 3 to 8, the ultrasonic emission surface 122 may have a variety of shapes such as a circular shape, a linear structure including a linear part and a semicircular part, and the like. Accordingly, a variety of arrangements of the ultrasonic generation unit 120 and the radio frequency generation unit 130 may be implemented.

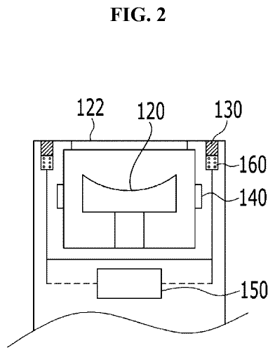

As shown in FIGS. 1 and 2, the ultrasonic generation unit 120 may be installed in the handpiece body 110, specifically, the cartridge portion, and generate an ultrasonic wave so as to emit the ultrasonic wave toward skin through the ultrasonic emission surface 122. In detail, the ultrasonic generation unit 120 may be a transducer configured to generate a focused ultrasonic wave and form a thermal lesion and may emit the focused ultrasonic wave toward the skin through the ultrasonic emission surface 122.

The above-described focused ultrasonic wave may be emitted toward thin skin tissue to be used for face lifting or skin tightening treatment and may be emitted toward a subcutaneous layer to be used for obesity treatment which burns or melts and splits fatty tissue.

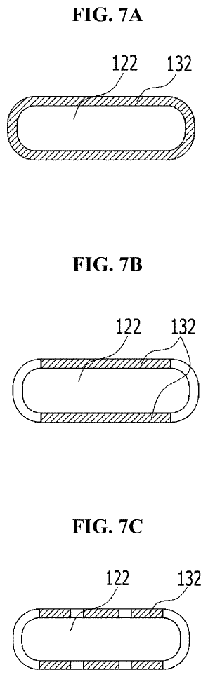

Accordingly, since a focal depth of the focused ultrasonic wave is set to be different according to a purpose of treatment, the cartridge portion including the ultrasonic generation unit set with a different focal depth may be replaced. As shown in FIGS. 3 and 7, the radio frequency generation unit 130 may be disposed at the one end of the handpiece body 110 on the periphery of the ultrasonic emission surface 122 and may generate a radio frequency. In other words, the radio frequency generation unit 130 may generate a radio frequency using a radio frequency electrode 132 and apply a radio frequency current to the skin.

The radio frequency may be classified according to definition thereof and may have a frequency range of 30 kHz to 300 MHz. The radio frequency may be applied to the skin so as to provide effects of skin rejuvenation, skin elasticity improvement, stimulation of blood and lymph circulation, and the like.

The radio frequency generation unit 130 may adjust the frequency of the radio frequency so as to adjust a depth at which transmitted radio frequency energy is focused. In detail, the radio frequency generation unit 130 may decrease the frequency of the radio frequency so as to transmit energy deeper.

The radio frequency generation unit 130 may set a depth at which energy is focused to be different by adjusting the frequency according to a purpose of treatment for face lifting, skin tightening treatment, or adipolysis treatment.

Accordingly, energy may be focused on a corium layer, a fascial plane, or a superficial musculo-aponeurotic system (SMAS) layer which is located at about 1.5 mm to 4.5 mm from a surface of the skin and a thermal lesion or thermal coagulation area may be formed so as to perform face lifting or skin tightening treatment. In order to form an energy-focused depth for face lifting or skin tightening treatment, in detail, a radio frequency at 1 MHz to 2 MHz may be applied.

Also, energy may be focused on a subcutaneous layer located at about 8 mm to 13 mm from the surface of the skin so as to perform adipolysis treatment of burning or melting fatty tissue. To form an energy-focused depth for the adipolysis treatment, in detail, a radio frequency at 300 kHz and 500 kHz may be applied.

When the radio frequency generation unit 130 adjusts the frequency to overlap the energy-focused depth with a focal depth of an ultrasonic wave generated by the ultrasonic generation unit 120, an effect of treatment may be increased and a treatment time may be reduced.

The radio frequency generation unit 130 may generate a radio frequency with multi-pulses a plurality of times for a short time. When the radio frequency is applied a plurality of times, as the number of application increases, resistance at an application area decreases. Accordingly, an energy loss may be reduced and energy may be focused so as to increase an effect of treatment.

Also, the cooling unit 140 may cool the radio frequency generation unit 130 heated by a radio frequency for a standby time before the radio frequency generation unit 130 generates the radio frequency with multi-pulses and generates a next radio frequency. Accordingly, skin damage caused by heat generated by the radio frequency generation unit 130 may be prevented.

Meanwhile, a skin cooling portion which will be described below may cool the skin for an intermediate standby time of the multi-pulses and prevent damage to the skin.

The radio frequency generation unit 130 may include a plurality of such radio frequency electrodes 132 arranged on the periphery of the ultrasonic emission surface 122. The number, arrangement, and the like of the radio frequency electrodes 132 may be formed to be different according to a shape of the ultrasonic emission surface 122. In more detail, the arrangement of the radio frequency electrodes 132 will be described below.

As shown in FIG. 2, the contact sensor 160 may be coupled to the one end of the handpiece body 110 so as to sense whether the one end of the handpiece body 110 comes into contact with the skin.

The contact sensor 160 may sense contact between the one end of the handpiece body 110 and the skin through a pressure, current, and the like, may sense whether contact with the skin is occurring, and may transmit a data signal thereof to the control unit 150 which will be described below. In the embodiment, for example, a capacitance sensor may be used as the contact sensor 160.

As shown in FIG. 2, when it is sensed by the contact sensor 160 that the one end of the handpiece body 110 comes into contact with the skin, the control unit 150 may receive a signal and control operations of the ultrasonic generation unit 120 and/or the radio frequency generation unit 130. Also, the control unit 150 may control an operation of the cooling unit 140 which will be descried below.

Also, the control unit 150 may adjust a frequency generated by the radio frequency generation unit 130 according to a focal length set in the ultrasonic generation unit when the cartridge portion is replaced. In detail, the control unit 150 may adjust the frequency of a radio frequency to overlap an ultrasonic focal depth with a radio frequency energy focused depth according to replacement of the cartridge portion so as to improve an effect of treatment and to reduce a treatment time.

Meanwhile, a printed circuit board 170 electrically connected to the radio frequency generation unit 130 and the contact sensor 160 and configured to control the same may be installed at the one end of the handpiece body 110.

Generally, the printed circuit board 170 includes an insulation substrate, and a plurality of holes may be formed to allow electrical components to be coupled and a circuit may be printed on a surface of a substrate.

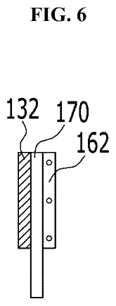

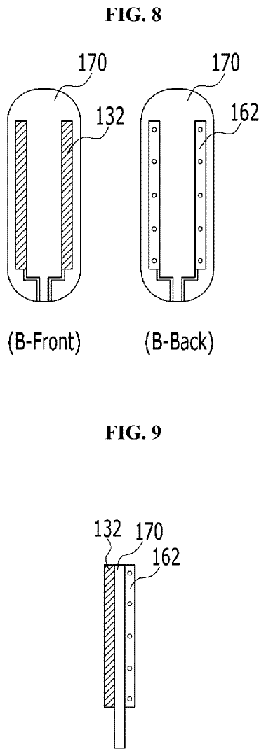

As shown in FIGS. 6 and 9, a contact sensor electrode 162 of the contact sensor 160 may be formed on one surface of the printed circuit board 170, and the radio frequency electrode 132 of the radio frequency generation unit 130 may be formed on the other surface thereof. In other words, the contact sensor 160 and the radio frequency electrode 132 may be coupled to be integrally formed on each surface of the printed circuit board 170.

As shown in FIGS. 4 and 8, the radio frequency electrode 132 may be disposed on the printed circuit board 170 at a position corresponding to the contact sensor electrode 162 in a plane.

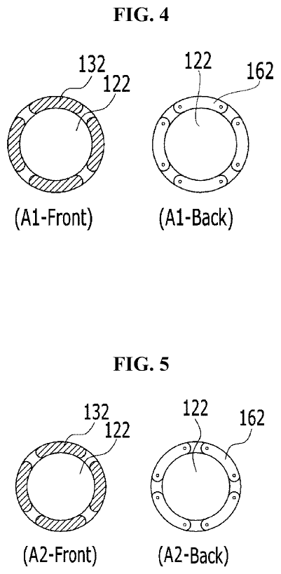

Unlike the above, as shown in FIG. 5, the radio frequency electrode 132 may be disposed to alternate with the contact sensor electrode 162. In detail, the radio frequency electrode 132 may be disposed at a position rotated from the position corresponding to the contact sensor electrode 162.

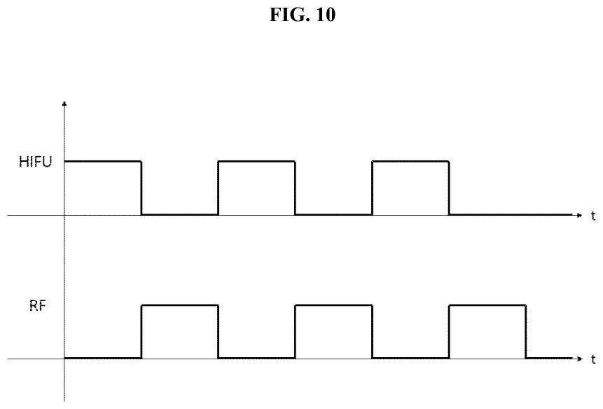

As shown in FIG. 10, the ultrasonic generation unit 120 and the radio frequency generation unit 130 may be alternately operated. In other words, in the beauty medical device 100 of the embodiment, while a practitioner grips and moves the handpiece body 110, the ultrasonic generation unit 120 and the radio frequency generation unit 130 may alternately operate so as to perform ultrasonic treatment and radio frequency treatment in one operation.

However, the ultrasonic generation unit 120 and the radio frequency generation unit 130 may be operated at the same time to perform adequate treatment on a person to be treated according to a treatment environment, a treatment method, and the like.

As shown in FIG. 2, the cooling unit 140 may be coupled to the inside of the handpiece body 110 to cool heat generated by at least one of the ultrasonic generation unit 120 and the radio frequency generation unit 130.

At least one of the ultrasonic generation unit 120 and the radio frequency generation unit 130 according to the present invention may generate heat while operating. The radio frequency electrode 132 of the radio frequency generation unit 130 of the embodiment may generate heat of about 60 degrees.

Not only may the heat generated by at least one of the ultrasonic generation unit 120 and the radio frequency generation unit 130 damage a component coupled to the handpiece body 110 but also the one end of the handpiece body 110 which is overheated during treatment may come into contact with the skin and damage the skin. To remedy the above problem, the cooling unit 140 may be installed on the handpiece body 110.

Also, when a temperature inside the handpiece body 110 exceeds a preset temperature, the control unit 150 may control the operation of the cooling unit 140.

In detail, when the temperature inside the handpiece body 110 increases due to heat caused by the operation of at least one of the ultrasonic generation unit 120 and the radio frequency generation unit 130, to prevent a problem caused by the overheating, the control unit 150 may operate the cooling unit 140 to lower the temperature inside the handpiece body 110 when the temperature inside the handpiece body 110 exceeds the preset temperature.

The skin cooling portion (not shown) may be formed on the cartridge portion and cool a treated region.

The skin cooling portion may spray a cooling gas for cooling the skin over the skin so as to reduce damage to the skin caused by the treatment. Also, the skin may be cooled by absorbing heat of the skin using a Peltier module or cooling water.

Subsequently, the beauty medical device 100 according to one embodiment of the present invention will be described with reference to FIGS. 3 to 6.

As shown in FIG. 3, the ultrasonic emission surface 122 may have a circular shape.

As shown in FIG. 3A1, the radio frequency generation unit 130 may include an annular radio frequency electrode 132 surrounding the ultrasonic emission surface 122. Also, as shown in FIGS. 3A2 and 3A3, the radio frequency generation unit 130 may include a plurality of radio frequency electrodes 132 arranged to be radially symmetrical on the basis of the ultrasonic emission surface 122.

As shown in FIG. 4, the radio frequency electrodes 132 and the contact sensor electrodes 162 may be arranged on both surfaces of the printed circuit board 170 at positions corresponding to each other. According to a circuit disposition design, as shown in FIG. 5, the radio frequency electrodes 132 and the contact sensor electrodes 162 may be arranged at alternating positions.

Subsequently, the beauty medical device 100 according to another embodiment of the present invention will be described with reference to FIGS. 7 to 9.

As shown in FIG. 7, the ultrasonic emission surface 122 may include a linear portion having a quadrangular shape and a pair of semicircular portions having a semicircular shape and disposed on both ends of the linear portion to be symmetrical to each other.

According to an operation method, the transducer of the ultrasonic generation unit 120 may emit an ultrasonic wave while moving along the linear portion of the ultrasonic emission surface 122 inside the cartridge portion.

As shown in FIG. 7B1, the radio frequency generation unit 130 may include an integrated radio frequency electrode 132 formed along the periphery of the ultrasonic emission surface 122. Also, as shown in FIGS. 7B2 and 7B3, the radio frequency generation unit 130 may include a plurality of radio frequency electrodes 132 arranged to be symmetrical on the basis of a virtual central line longitudinally crossing the ultrasonic emission surface 122.

The radio frequency electrodes 132 and the contact sensor electrodes 162 may be arranged at positions corresponding to each other as shown in FIGS. 8 and 9.

Although the embodiments of the present invention have been described above, it should be noted that one of ordinary skill in the art may make a variety of modifications and changes of the present invention by adding, changing, eliminating, inserting, or the like of components without departing from the concept of the present invention which is disclosed in the claims and this will be included within the scope of the present invention.

1. A beauty medical device comprising:
a handpiece body grippable by a practitioner for a beauty medical treatment;
a cartridge detachably coupled to an end of the handpiece body and having a distal end for contacting a surface of a skin for the beauty medical treatment;
a transducer configured to generate a focused ultrasound at a selected depth to form a thermal lesion or a thermal coagulation area in the skin;
radio frequency electrodes configured to apply radio frequency current to the skin;
an ultrasonic emission surface provided on the distal end of the cartridge,
wherein the transducer is configured to emit the focused ultrasound through the ultrasonic emission surface to the skin and the radio frequency electrodes are provided on the distal end of the cartridge to be arranged on periphery of the ultrasonic emission surface so that the ultrasonic emission surface and the radio frequency electrodes are simultaneously come into contact with the skin according to contacting the distal end of the cartridge with the surface of the skin for treatment;
a contact sensor configured to sense whether the radio frequency electrodes come into contact with the surface of the skin; and
a controller configured to control the radio frequency electrodes to apply radio frequency current to form a radio frequency energy field in the skin and to control the transducer to emit the focused ultrasound through the ultrasonic emission surface at the selected depth in the radio frequency energy field in the skin to form the thermal lesion or the thermal coagulation area in the skin according to determining the ultrasonic emission surface and the radio frequency electrodes simultaneously contact with the skin surface as a sensing result of the contact sensor.

2. The device according to claim 1, wherein the contact sensor includes a contact sensor electrode formed on one side of a printed circuit board provided in the cartridge, wherein the radio frequency electrodes are formed on the other side of the printed circuit board to be electrically connected to the contact sensor electrode so that the contact sensor is able to sense through the contact sensor electrode according to contacting the radio frequency electrodes with the skin surface.

3. The device according to claim 1, wherein the contact sensor includes a capacitance sensor configured to sense a change of capacitance according to the skin contact of the radio frequency electrodes.

4. The device according to claim 1, wherein the controller controls to apply radio frequency current through the skin-contacted radio frequency electrodes with multi-pulses a predetermined plurality of times so that the number of radio frequency application increases, resistance at an application area of the skin decreases, and then the controller controls the transducer to emit the focused ultrasound through the ultrasonic emission surface to form the thermal lesion or the thermal coagulation area in the resistance-decreased area of the skin.

5. The device according to claim 1, further comprising a cooler configured to cool the radio frequency electrodes heated by the applied radio frequency current for a standby time before a next applying radio frequency current.

6. The device according to claim 1, further comprising a skin cooler configured to form on the distal end of the cartridge so as to cool an area treated by the radio frequency electrodes on the skin for an intermediate standby time of multi-pulses of radio frequency current of the radio frequency electrodes.

7. The device according to claim 1, further comprising a skin cooler configured to spray a cooling gas for cooling an area treated by the radio frequency electrodes on the skin.

8. The device according to claim 1, wherein the controller controls to apply radio frequency current with pulses having predetermined intervals and to emit the focused ultrasound with pulses having intervals alternate with the radio frequency pulses.
